(19) United States
(12) Patent Application Publication
(10) Pub. No.: US 2023/0191084 A1
(43) Pub. Date: Jun. 22, 2023

(54) Steerable Cannulas and Hinged or Steerable Needles Including Access Sets and Methods Thereof

(71) Applicant: Becton, Dickinson and Company, Franklin Lakes, NJ (US)

(72) Inventors: Xiaowen Sun, Shanghai (CN); Luke X. Zeng, Suzhou (CN); Peng Yi, Shanghai (CN); Zhixiu He, Shanghai (CN); Yuran Hua, Shanghai (CN)

(21) Appl. No.: 17/917,222

(22) PCT Filed: Apr. 8, 2020
(86) PCT No.: PCT/US2020/027333

§ 371 Date: Oct. 5, 2022

(51) Int. Cl. A61M 25/06; A61M 25/00

(52) U.S. Cl. CPC A61M 25/0606; A61M 25/0097; A61M 25/0136

(57) ABSTRACT

Disclosed are steerable cannulas and hinged or steerable needles including access sets and methods thereof. A steerable cannula can include a compound tube, a cannula tip, a compound hub, and an articulation mechanism. A distal-end portion of the compound tube can include a flexible hinge for adjusting a cannula angle between proximal and distal portions of the compound tube adjacent the flexible hinge. A cannula tip coupled to a distal end of the compound tube can include a tip opening fluidly connected to a lumen of the compound tube. The compound hub is disposed about a proximal-end portion of the compound tube. The articulation mechanism is controlled by at least a rotatable element of the compound hub for adjusting the cannula angle. The articulation mechanism is configured to longitudinally move an inner tube of the compound tube relative to an outer tube to adjust the cannula angle.

### BACKGROUND

In a healthy person, blood flowing from the stomach, esophagus, or intestines first flows through the liver. In an unhealthy person having, for example, liver damage, there can be blood flow-restricting blockages in the liver such that blood cannot easily flow therethrough. Such a condition is known as portal hypertension. Common causes of portal hypertension include alcohol abuse, too much iron in the liver (e.g., hemochromatosis), hepatitis B, hepatitis C, or blood clots in a vein that flows from the liver to the heart. When portal hypertension occurs, the blood flow-restricting blockages can elevate pressure in the portal vein causing it to rupture and seriously bleed. A person with portal hypertension can also have bleeding from the veins of the stomach, esophagus, or intestines (e.g., variceal bleeding), a buildup of fluid in the belly (e.g., ascites), or a buildup of fluid in the chest (e.g., hydrothorax).

Portal hypertension is often treated by way of a percutaneous procedure involving placement of a transjugular intrahepatic portosystemic shunt (“TIPS”) between the hepatic vein and the portal vein as shown in FIG. 15 to establish blood flow through the liver. Placement of a portosystemic shunt between the right hepatic vein and the right portal vein is generally preferred, and transjugular liver access sets currently available are generally dedicated to this end. For example, bent or curved stiffening cannulas are often provided in the foregoing transjugular liver access sets to support an anterior needle throw from the right hepatic vein to the right portal vein. Being that the stiffening cannulas are already bent or curved, the stiffening cannulas do not easily accommodate anatomical variations in patients or different modes of access within the liver such as from the middle hepatic vein to either the left or right portal vein, which respectively requires either an anterior or posterior needle throw, or the left hepatic vein to the left portal vein, which requires a posterior needle throw. In view of the foregoing, the transjugular liver access sets currently available can inadvertently contribute to prolonged procedures and decreased success rates for patients having anatomical variations or needing different modes of access within the liver.

Disclosed herein are steerable cannulas and hinged or steerable needles including access sets and methods thereof that address at least the forgoing shortcomings.

### SUMMARY

Disclosed herein is a steerable cannula for a TIPS procedure. The steerable cannula includes, in some embodiments, an elongate compound tube, a cannula tip, a compound hub, and an articulation mechanism. The compound tube has a proximal-end portion and a distal-end portion. The distal-end portion includes a flexible hinge for adjusting a cannula angle between proximal and distal portions of the compound tube adjacent the flexible hinge. The cannula tip is coupled to a distal end of the compound tube. The cannula tip includes a tip opening fluidly connected to a lumen of the compound tube. The compound hub is disposed about the proximal-end portion of the compound tube. The articulation mechanism is controlled by at least a rotatable element of the compound hub for adjusting the cannula angle. The articulation mechanism is configured to longitudinally move an inner tube of the compound tube relative to an outer tube of the compound tube to adjust the cannula angle.

In some embodiments, each tube of the inner tube and the outer tube of the compound tube includes a set of transverse cutouts. The flexible hinge of the compound tube is formed between offset sets of the transverse cutouts in at least a non-deflected state of the compound tube.

In some embodiments, the rotatable element of the compound hub is a rotatable handle configured to rotate relative to a housing of the compound hub. The rotatable handle includes a threaded bore or an insert including a threaded bore configured to drive a threaded plug proximally when the rotatable handle is rotated in a first direction or distally when the rotatable handle is rotated in a second direction.

In some embodiments, the threaded plug is fixedly attached to the inner tube of the compound tube such that the inner tube is driven proximally or distally in concert with the threaded plug when the rotatable handle is respectively rotated in the first direction or the second direction.

In some embodiments, the outer tube of the compound tube is fixedly attached to a stationary element disposed in the housing of the compound hub. The inner tube of the compound tube passes through the stationary element.

In some embodiments, a distal end of each tube of the inner tube and the outer tube of the compound tube is fixedly attached to the cannula tip. This allows the inner tube to be longitudinally moved relative to the outer tube when the rotatable handle of the compound hub is rotated in the first direction or the second direction.

In some embodiments, the compound tube includes a needle lumen longitudinally extending through the compound tube configured for disposing a needle or a catheter-and-needle assembly therein.

In some embodiments, the cannula angle ranges from about −90° to about 90°.

In some embodiments, the compound hub includes a female Luer connector extending through the rotatable element and into a distal-end portion of the inner tube of the compound tube. The Luer connector is configured to fluidly connect the lumen of the compound tube to another medical device.

In some embodiments, the steerable cannula is configured to stiffen a catheter assembly in support of a needle throw from the catheter assembly at the cannula angle chosen for the needle throw.

Also disclosed herein is a hinged needle for a TIPS procedure. The hinged needle includes, in some embodiments, an elongate needle tube, a pencil-point needle tip, and a hub. The elongate needle tube has a proximal-end portion and a distal-end portion. The distal-end portion includes a flexible hinge configured to curve when the hinged needle is inserted into a curved channel. The needle tip is coupled to a distal end of the needle tube. The needle tip includes a tip opening fluidly connected to a lumen of the needle tube. The hub is around the proximal-end portion of the needle tube. The hub includes a hub opening fluidly connected to the lumen of the needle tube.

In some embodiments, an entirety of the needle tube is a metal hypotube.

In some embodiments, the flexible hinge is a spiral-cut or slotted portion of the hypotube.

In some embodiments, at least the flexible hinge of the hypotube is nitinol.

In some embodiments, the needle tube is a hypotube coupled to a spring as the flexible hinge.

In some embodiments, the needle tube is a hypotube coupled to a braided tube as the flexible hinge.

In some embodiments, the tip opening of the needle tip is distal to where the needle tip is coupled to the distal end of the needle tube.

In some embodiments, the hub includes a female Luer connector in a proximal-end portion of the hub. The Luer connector is configured for fluidly connecting the lumen of the needle tube to another medical device.

In some embodiments, the needle tip is configured to pierce a liver parenchyma and a branch of a portal vein.

In some embodiments, the hinged needle is configured to provide instant blood flashback when the needle tip is properly placed in at least the branch of the portal vein.

Also disclosed herein is an access set for a TIPS procedure. The access set includes, in some embodiments, an introducer sheath, a stiffening cannula, and a piercing device. The stiffening cannula is selected from a steerable cannula and a fixed-curve cannula, each of which is configured to support a piercing-device throw therefrom. The steerable cannula includes an articulation mechanism configured to longitudinally move an inner tube of an elongate compound tube relative to an outer tube of the compound tube to adjust a piercing-device angle for the piercing-device throw. The piercing device is selected from a hinged needle, a Colapinto needle, and a trocar stylet. The hinged needle includes an elongate needle tube having a flexible hinge and a pencil-point needle tip coupled to a distal end of the needle tube. The needle tip has a tip opening fluidly connected to a lumen of the needle tube. The access set includes at least the steerable cannula or the hinged needle.

In some embodiments, each tube of the inner tube and the outer tube of the compound tube of the steerable cannula includes a set of transverse cutouts forming a flexible hinge of the compound tube. Each set of transverse cutouts is offset from the other in at least a non-deflected state of the compound tube.

In some embodiments, a rotatable handle of a compound hub of the steerable cannula is configured to rotate relative to a housing of the compound hub. The rotatable handle includes a threaded bore or an insert including a threaded bore configured to drive a threaded plug proximally when the rotatable handle is rotated in a first direction or distally when the rotatable handle is rotated in a second direction.

In some embodiments, the threaded plug is fixedly attached to the inner tube of the compound tube such that the inner tube is driven proximally or distally in concert with the threaded plug when the rotatable handle is respectively rotated in the first direction or the second direction.

In some embodiments, the outer tube of the compound tube is fixedly attached to a stationary element disposed in the housing of the compound hub. The inner tube of the compound tube passes through the stationary element.

In some embodiments, a distal end of each tube of the inner tube and the outer tube of the compound tube is fixedly attached to a cannula tip. This allows the inner tube to be longitudinally moved relative to the outer tube when the rotatable handle of the compound hub is rotated in the first direction or the second direction.

In some embodiments, an entirety of the needle tube of the hinged needle is a metal hypotube. The flexible hinge is selected from a spiral-cut portion of the hypotube, a slotted portion of the hypotube, and a nitinol portion of the hypotube.

In some embodiments, the needle tube of the hinged needle is a hypotube coupled to a spring or braided tube as the flexible hinge.

In some embodiments, the tip opening of the needle tip of the hinged needle is distal to where the needle tip is coupled to the distal end of the needle tube.

In some embodiments, the access set further includes an access device and a first catheter. The access device is configured to create an access site in at least a jugular vein. The first catheter is configured to catheterize an inferior vena cava from the access site.

In some embodiments, the access set further includes a dilator for combination with the introducer sheath in a first assembly. The introducer sheath is configured to accept the dilator in a lumen of the introducer sheath such that a distal-end portion of the dilator extends past a distal end of the introducer sheath for dilation of a piercing-device tract.

In some embodiments, the access set further includes a second catheter for combination with the stiffening cannula in a second assembly. The second catheter is configured to accept the stiffening cannula in a lumen of the second catheter and conceal a distal end of the stiffening cannula with a distal-end portion of the second catheter. This protects other components of the access set from being damaged by the distal end of the stiffening cannula when advancing the second assembly in vivo.

In some embodiments, the access set further includes a third catheter for combination with the piercing device in a third assembly. The third catheter is configured to accept the piercing device in a lumen of the third catheter and conceal a distal end of the piercing device with a distal-end portion of the third catheter. This protects other components of the access set from being pierced by the distal end of the piercing device when advancing the third assembly in vivo.

Also disclosed herein is a steerable needle for a TIPS procedure. The steerable needle includes, in some embodiments, an elongate compound tube, a needle tip, and a compound hub, and an articulation mechanism. The elongate compound tube has a proximal-end portion and a distal-end portion. The distal-end portion includes a flexible hinge for adjusting a needle angle between proximal and distal portions of the compound tube adjacent the flexible hinge. The needle tip is coupled to a distal end of the compound tube. The needle tip includes a tip opening fluidly connected to a lumen of the compound tube. The compound hub is disposed about the proximal-end portion of the compound tube. The articulation mechanism is controlled by at least a rotatable element of the compound hub for adjusting the needle angle. The articulation mechanism is configured to longitudinally move an inner tube of the compound tube relative to an outer tube of the compound tube to adjust the needle angle.

In some embodiments, each tube of the inner tube and the outer tube of the compound tube includes a set of transverse cutouts. The flexible hinge of the compound tube is formed between offset sets of the transverse cutouts in at least a non-deflected state of the compound tube.

In some embodiments, the rotatable element of the compound hub is a rotatable handle configured to rotate relative to a fixed handle of the compound hub. The rotatable handle includes a threaded bore configured to drive a threaded plug proximally when the rotatable handle is rotated in a first direction or distally when the rotatable handle is rotated in a second direction.

In some embodiments, the threaded plug is fixedly attached to the inner tube of the compound tube such that the inner tube is driven proximally or distally in concert with the threaded plug when the rotatable handle is respectively rotated in the first direction or the second direction.

In some embodiments, the outer tube of the compound tube is fixedly attached to the fixed handle of the compound hub. The inner tube of the compound tube passes through the fixed handle.

In some embodiments, a distal end of each tube of the inner tube and the outer tube of the compound tube is fixedly attached to the needle tip. This allows the inner tube to be longitudinally moved relative to the outer tube when the rotatable handle of the compound hub is rotated in the first direction or the second direction.

In some embodiments, the compound hub includes a female Luer connector disposed in the rotatable element over a tubular extension of a distal-end portion of the threaded plug. The Luer connector is configured to fluidly connect the lumen of the compound tube to another medical device.

In some embodiments, the compound tube includes a lumen longitudinally extending through the compound tube configured for aspirating blood or injecting contrast.

In some embodiments, the needle angle ranges from about −90° to about 90° for a needle throw from a catheter assembly at the needle angle chosen for the needle throw.

In some embodiments, the needle tip is configured to pierce a liver parenchyma and a branch of a portal vein.

In some embodiments, the steerable needle is configured to provide instant blood flashback when the needle tip is properly placed in at least the branch of the portal vein.

Also disclosed herein is another access set for a TIPS procedure. The access set includes, in some embodiments, an introducer sheath and a steerable needle. The steerable needle is configured for a needle throw from the introducer sheath. The steerable needle includes an articulation mechanism configured to longitudinally move an inner tube of an elongate compound tube relative to an outer tube of the compound tube to adjust a needle angle for the needle throw.

In some embodiments, each tube of the inner tube and the outer tube of the compound tube of the steerable needle includes a set of transverse cutouts forming a flexible hinge of the compound tube. Each set of transverse cutouts is offset from the other in at least a non-deflected state of the compound tube.

In some embodiments, a rotatable handle of a compound hub is configured to rotate relative to a fixed handle of the compound hub. The rotatable handle includes a threaded bore configured to drive a threaded plug proximally when the rotatable handle is rotated in a first direction or distally when the rotatable handle is rotated in a second direction.

In some embodiments, the threaded plug is fixedly attached to the inner tube of the compound tube such that the inner tube is driven proximally or distally in concert with the threaded plug when the rotatable handle is respectively rotated in the first direction or the second direction.

In some embodiments, the outer tube of the compound tube is fixedly attached to the fixed handle of the compound hub. The inner tube of the compound tube passes through the fixed handle.

In some embodiments, a distal end of each tube of the inner tube and the outer tube of the compound tube is fixedly attached to a needle tip including a tip opening fluidly connected to a lumen of the compound tube. This allows the inner tube to be longitudinally moved relative to the outer tube when the rotatable handle of the compound hub is rotated in the first direction or the second direction.

In some embodiments, the access set further includes an access device and a first catheter. The access device is configured to create an access site in at least a jugular vein. The first catheter is configured to catheterize an inferior vena cava from the access site.

In some embodiments, the access set further includes a dilator for combination with the introducer sheath in a first assembly. The introducer sheath is configured to accept the dilator in a lumen of the introducer sheath such that a distal-end portion of the dilator extends past a distal end of the introducer sheath for dilation of a needle tract.

In some embodiments, the access set further includes a third catheter for combination with the steerable needle in a third assembly. The third catheter is configured to accept the steerable needle in a lumen of the third catheter and conceal a distal end of the steerable needle with a distal-end portion of the third catheter. This protects other components of the access set from being pierced by the distal end of the steerable needle when advancing the third assembly in vivo.

Also disclosed herein is a method for a TIPS procedure. The method includes, in some embodiments, a first cannula-inserting step, a first piercing device-inserting step, a cannula-articulating step, a cannula angle-choosing step, and a piercing device-throwing step. The first cannula-inserting step includes inserting a steerable cannula into an introducer sheath positioned in a distal portion of a hepatic vein. The steerable cannula includes an articulation mechanism configured to longitudinally move an inner tube of an elongate compound tube relative to an outer tube of the compound tube to adjust a cannula angle for a piercing-device throw. The first piercing device-inserting step includes inserting a piercing device into the steerable cannula. The cannula-articulating step includes articulating the steerable cannula by rotating a rotatable handle of a compound hub of the steerable cannula either before or after inserting the piercing device into the steerable cannula. The cannula angle-choosing step includes choosing the cannula angle for the piercing-device throw from the hepatic vein to a portal vein. The piercing device-throwing step includes throwing the piercing device from the hepatic vein to the portal vein in accordance with the cannula angle and with support provided by the compound tube.

In some embodiments, the method further includes a second cannula-inserting step. The second cannula-inserting step includes inserting the steerable cannula into a second catheter to form a second catheter assembly before inserting the steerable cannula into the introducer sheath. The steerable cannula is inserted into the introducer sheath as part of the second catheter assembly.

In some embodiments, the method further includes a second piercing device-inserting step. The second piercing device-inserting step includes inserting the piercing device into a third catheter to form a third catheter assembly before inserting the piercing device into the steerable cannula. The piercing device is inserted into the steerable cannula as part of the third catheter assembly.

In some embodiments, the piercing device-throwing step includes throwing the piercing device from a right hepatic vein to a right portal vein with an anterior piercing-device throw, a middle hepatic vein to either a left portal vein or the right portal vein respectively with either a different anterior piercing-device throw or a posterior piercing-device throw, or a left hepatic vein to the left portal vein with a different posterior piercing-device throw. The piercing device-throwing step is in accordance with the cannula angle chosen for the piercing-device throw.

Also disclosed herein is another method for a TIPS procedure. The method includes, in some embodiments, a first cannula-inserting step, a first needle-inserting step, a cannula-orienting step, and a needle-throwing step. The first cannula-inserting step includes inserting a stiffening cannula into an introducer sheath positioned in a distal portion of a hepatic vein. The stiffening cannula is configured to support a needle throw from the stiffening cannula. The first needle-inserting step includes inserting a hinged needle into the stiffening cannula. The hinged needle including an elongate needle tube having a flexible hinge and a pencil-point needle tip coupled to a distal end of the needle tube. The needle tip has a tip opening fluidly connected to a lumen of the needle tube. The cannula-orienting step includes orienting the stiffening cannula toward a portal vein. The needle-throwing step includes throwing the hinged needle from the hepatic vein to the portal vein in accordance with an orientation of the stiffening cannula.

In some embodiments, the method further includes a syringe-connecting step, a plunger-pulling step, and a portal vein-confirming step. The syringe-connecting step includes connecting a tip of a syringe having a barrel filled with a contrast medium to a hub of the hinged needle. The plunger-pulling step includes pulling a plunger of the syringe to create a vacuum. The portal vein-confirming step includes slowly withdrawing the hinged needle until blood is seen in the barrel of the syringe. The portal vein-confirming step confirms the needle tip of the hinged needle is in the portal vein.

In some embodiments, the method further includes a second cannula-inserting step. The second cannula-inserting step includes inserting the stiffening cannula into a second catheter to form a second catheter assembly before inserting the stiffening cannula into the introducer sheath. The stiffening cannula is inserted into the introducer sheath as part of the second catheter assembly.

In some embodiments, the method further includes a second needle-inserting step. The second needle-inserting step includes inserting the hinged needle into a third catheter to form a third catheter assembly before inserting the hinged needle into the stiffening cannula. The hinged needle is inserted into the stiffening cannula as part of the third catheter assembly.

In some embodiments, the needle-throwing step includes throwing the hinged needle from a right hepatic vein to a right portal vein with an anterior needle throw, a middle hepatic vein to either a left portal vein or the right portal vein respectively with either a different anterior needle throw or a posterior needle throw, or a left hepatic vein to the left portal vein with a different posterior needle throw. The needle-throwing step is in accordance with the orientation of the stiffening cannula.

Also disclosed herein is another method for a TIPS procedure. The method includes, in some embodiments, a first needle-inserting step, a needle-articulating step, a needle angle-choosing step, and a needle-throwing step. The first needle-inserting step includes inserting a steerable needle into an introducer sheath positioned in a distal portion of a hepatic vein. The steerable needle includes an articulation mechanism configured to longitudinally move an inner tube of an elongate compound tube relative to an outer tube of the compound tube to adjust a needle angle for a needle throw. The needle-articulating step includes articulating the steerable needle by rotating a rotatable handle of a compound hub of the steerable needle either before or after inserting the steerable needle into the introducer sheath. The needle angle-choosing step includes choosing the needle angle for a needle throw from the hepatic vein to a portal vein. The needle-throwing step include throwing the steerable needle from the hepatic vein to the portal vein in accordance with the needle angle.

In some embodiments, the method further includes a syringe-connecting step, a plunger-pulling step, and a portal vein-confirming step. The syringe-connecting step includes connecting a tip of a syringe having a barrel filled with a contrast medium to a Luer connector of the steerable needle. The plunger-pulling step includes pulling a plunger of the syringe to create a vacuum. The portal vein-confirming step includes slowly withdrawing the steerable needle until blood is seen in the barrel of the syringe. The portal vein-confirming step confirms a needle tip of the steerable needle is in the portal vein.

In some embodiments, the method further includes a second needle-inserting step. The second needle-inserting step includes inserting the steerable needle into a third catheter to form a third catheter assembly before inserting the steerable needle into the introducer sheath. The steerable needle is inserted into the introducer sheath as part of the third catheter assembly.

In some embodiments, the needle-throwing step includes throwing the steerable needle from a right hepatic vein to a right portal vein with an anterior needle throw, a middle hepatic vein to either a left portal vein or the right portal vein respectively with either a different anterior needle throw or a posterior needle throw, or a left hepatic vein to the left portal vein with a different posterior needle throw, in accordance with the needle angle.

These and other features of the concepts provided herein will become more apparent to those of skill in the art in view of the accompanying drawings and following description, which disclose particular embodiments of such concepts in greater detail.

### DRAWINGS

FIG. 1 illustrates a steerable cannula in accordance with some embodiments.

FIG. 2 illustrates a flexible hinge of a compound tube of the steerable cannula in accordance with some embodiments.

FIG. 3 illustrates a cross section of a compound hub of the steerable cannula in accordance with some embodiments.

FIG. 4 illustrates an articulation mechanism of the steerable cannula in accordance with some embodiments.

FIG. 5 further illustrates the articulation mechanism of the steerable cannula in accordance with some embodiments.

FIG. 6 illustrates an access set including the steerable cannula in accordance with some embodiments.

FIG. 7 illustrates a cross section of a hinged needle in accordance with some embodiments.

FIG. 8 illustrates a cross section of a needle tip of the hinged needle in accordance with some embodiments.

FIG. 9 illustrates an access set including the hinged needle and the steerable cannula in accordance with some embodiments.

FIG. 10 illustrates an assembly of the hinged needle disposed in the steerable cannula in accordance with some embodiments.

FIG. 11 illustrates a steerable needle in accordance with some embodiments.

FIG. 12A illustrates a flexible hinge of a compound tube of the steerable needle in accordance with some embodiments.

FIG. 12B illustrates the flexible hinge of the compound tube of the steerable needle having an alternative needle tip in accordance with some embodiments.

FIG. 13 illustrates an articulation mechanism of the steerable needle in accordance with some embodiments.

FIG. 14 illustrates an access set including the steerable needle in accordance with some embodiments.

FIG. 15 illustrates a percutaneous procedure involving placement of a TIPS between a hepatic vein and a portal vein in accordance with some embodiments.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

With respect to “proximal,” a “proximal portion” or a “proximal-end portion” of, for example, a catheter disclosed herein includes a portion of the catheter intended to be near a clinician when the catheter is used on a patient. Likewise, a “proximal length” of, for example, the catheter includes a length of the catheter intended to be near the clinician when the catheter is used on the patient. A “proximal end” of, for example, the catheter includes an end of the catheter intended to be near the clinician when the catheter is used on the patient. The proximal portion, the proximal-end portion, or the proximal length of the catheter can include the proximal end of the catheter; however, the proximal portion, the proximal-end portion, or the proximal length of the catheter need not include the proximal end of the catheter. That is, unless context suggests otherwise, the proximal portion, the proximal-end portion, or the proximal length of the catheter is not a terminal portion or terminal length of the catheter.

With respect to “distal,” a “distal portion” or a “distal-end portion” of, for example, a catheter disclosed herein includes a portion of the catheter intended to be near or in a patient when the catheter is used on the patient. Likewise, a “distal length” of, for example, the catheter includes a length of the catheter intended to be near or in the patient when the catheter is used on the patient. A “distal end” of, for example, the catheter includes an end of the catheter intended to be near or in the patient when the catheter is used on the patient. The distal portion, the distal-end portion, or the distal length of the catheter can include the distal end of the catheter; however, the distal portion, the distal-end portion, or the distal length of the catheter need not include the distal end of the catheter. That is, unless context suggests otherwise, the distal portion, the distal-end portion, or the distal length of the catheter is not a terminal portion or terminal length of the catheter.

Unless defined otherwise, all technical and scientific terms used herein have the same meaning as commonly understood by those of ordinary skill in the art.

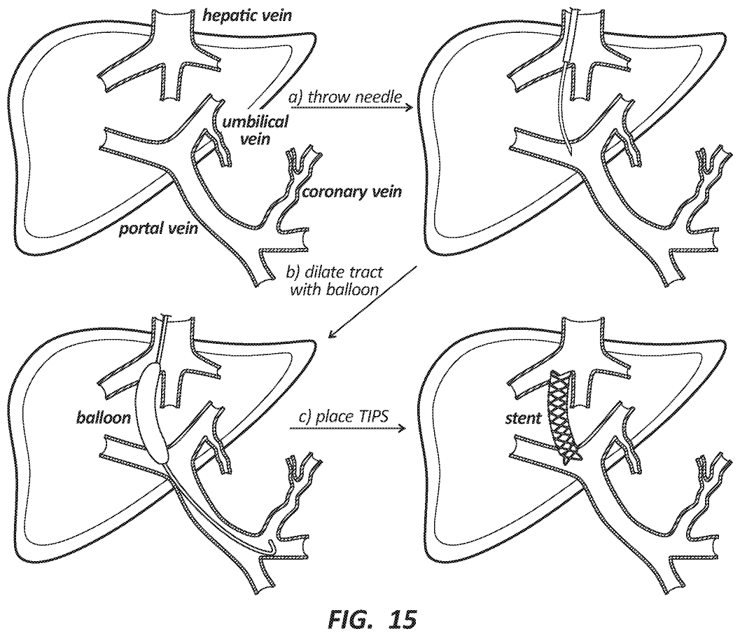

As set forth above, portal hypertension is often treated by way of a percutaneous procedure involving placement of a TIPS between the hepatic vein and the portal vein as shown in FIG. 15 to establish blood flow through the liver. Placement of a portosystemic shunt between the right hepatic vein and the right portal vein is generally preferred, and transjugular liver access sets currently available are generally dedicated to this end. For example, bent or curved stiffening cannulas are often provided in the foregoing transjugular liver access sets to support an anterior needle throw from the right hepatic vein to the right portal vein. Being that the stiffening cannulas are already bent or curved, the stiffening cannulas do not easily accommodate anatomical variations in patients or different modes of access within the liver such as from the middle hepatic vein to either the left or right portal vein, which respectively requires either an anterior or posterior needle throw, or the left hepatic vein to the left portal vein, which requires a posterior needle throw. In view of the foregoing, the transjugular liver access sets currently available can inadvertently contribute to prolonged procedures and decreased success rates for patients having anatomical variations or needing different modes of access within the liver.

Disclosed herein are steerable cannulas and hinged or steerable needles including access sets and methods thereof that address at least the forgoing shortcomings. While the steerable cannulas, hinged or steerable needles, access sets, and methods described herein are described in relation to TIPS procedures, it should be understood the steerable cannulas, hinged or steerable needles, access sets, and methods are not limited to TIPS procedures.

### Steerable Cannulas

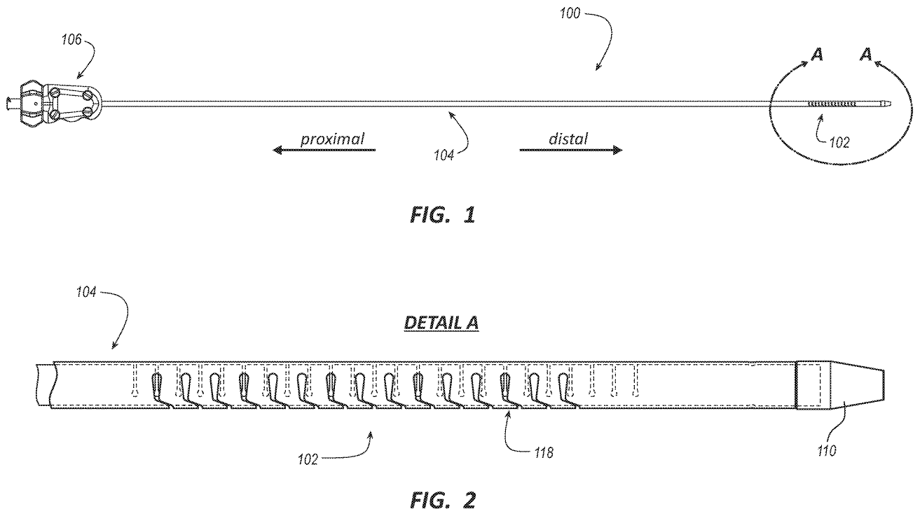

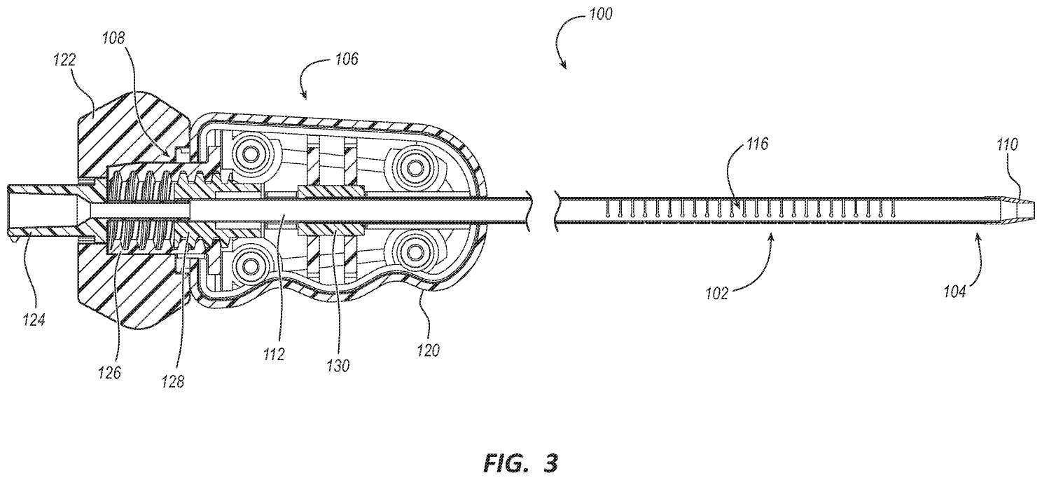

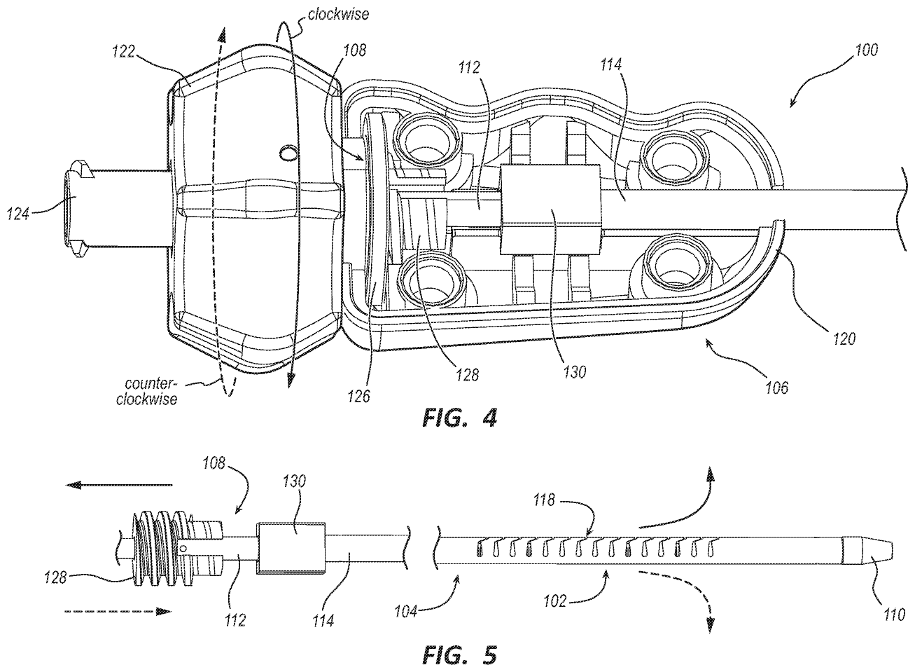

FIG. 1 illustrates a steerable cannula 100 in accordance with some embodiments. FIG. 2 illustrates a flexible hinge 102 of an elongate compound tube 104 of the steerable cannula 100 in accordance with some embodiments. FIG. 3 illustrates a cross section of a compound hub 106 of the steerable cannula 100 in accordance with some embodiments. FIG. 4 illustrates an articulation mechanism 108 of the steerable cannula 100 in accordance with some embodiments. FIG. 5 further illustrates the articulation mechanism 108 of the steerable cannula 100 in accordance with some embodiments.

The steerable cannula 100 includes, in some embodiments, the compound tube 104, a cannula tip 110, the compound hub 106, and the articulation mechanism 108. Each component of the compound tube 104, the cannula tip 110, the compound hub 106, and the articulation mechanism 108 is described in turn below. Due to the articulation mechanism 108 being formed between different elements of the compound tube 104, the cannula tip 110, and the compound hub 106, some elements of the compound tube 104, the cannula tip 110, and the compound hub 106 are described with respect to the articulation mechanism 108.

The compound tube 104 includes an inner tube 112, an outer tube 114, a lumen extending through an entirety of the compound tube 104, and the flexible hinge 102 in a distal-end portion of the compound tube 104.

The lumen of the compound tube 104 is configured for disposing a piercing device (e.g., a needle, a trocar stylet, etc.) or a catheter-and-piercing-device assembly (e.g., catheter-and-needle assembly, catheter-and-trocar-stylet, etc.) assembly therein in support of a piercing-device throw (e.g., a needle throw, a trocar-stylet throw, etc.) from the steerable cannula 100. The lumen of the compound tube 104 is best seen in FIG. 3, which includes a cross section of the compound tube 104.

The flexible hinge 102 of the compound tube 104 is configured for adjusting a cannula angle between proximal and distal portions of the compound tube 104 adjacent the flexible hinge 102 in support of a piercing-device throw from the steerable cannula 100. The cannula angle of the steerable cannula 100 can be adjusted to any angle between about −90° and 90°, including about 0° and 90°, such as about 0° and 45°, for example, about 0° and 35° for such a piercing-device throw.

The flexible hinge 102 of the compound tube 104 is formed between offset sets of transverse cutouts 116 and 118 in at least a non-deflected state of the compound tube 104. Each tube of the inner tube 112 and the outer tube 114 of the compound tube 104 includes a set of the cutouts, respectively the set of cutouts 116 and the set of cutouts 118. The set of cutouts 116 in the inner tube 112 is best seen in FIG. 3, and the set of cutouts 118 in the outer tube 114 is best seen in FIGS. 2 and 5. Each set of the sets of cutouts 116 and 118 can be laser cutouts. The laser cutouts are precise cutouts characterized by having edges with high-quality surface finishes.

The cannula tip 110 is coupled to a distal end of the compound tube 104. A distal end of each tube of the inner tube 112 and the outer tube 114 of the compound tube 104 is fixedly attached to the cannula tip 110. The cannula tip 110 includes a tip opening fluidly connected to the lumen of the compound tube 104. The tip opening in the cannula tip 110 is best seen in FIG. 3, which includes a cross section of the cannula tip 110.

The compound hub 106 includes a housing 120, a rotatable element 122 (e.g., a rotatable handle), and a Luer connector 124. The compound hub 106 is disposed about a proximal-end portion of the compound tube 104.

The housing 120 of the compound hub 106 can be formed of housing halves coupled together. FIG. 4 illustrates internal elements of the compound hub 106 in accordance with removal of one half of the housing halves from the housing 120.

The rotatable element 122 of the compound hub 106 is configured to rotate relative to the housing 120 of the compound hub 106. As best shown in FIG. 3, the rotatable element 122 includes a non-threaded bore and a concentric insert 126 therein including a threaded bore having internal threads configured to drive a threaded plug 128 having external threads proximally when the rotatable element 122 is rotated in a first direction (e.g., clockwise) or distally when the rotatable element 122 is rotated in a second direction (e.g., counterclockwise). The concentric insert 126 includes a flanged end configured to keep a combination of the rotatable element 122 and the concentric insert 126 captive by the housing 120. Alternatively, the rotatable element 122 includes a threaded bore having internal threads configured to drive the threaded plug 128 having the external threads proximally when the rotatable element 122 is rotated in the first direction or distally when the rotatable element 122 is rotated in the second direction. Such a rotatable element can, itself, include a flanged end configured to keep the rotatable element 122 captive by the housing 120.

The Luer connector 124 is a female Luer connector extending through the rotatable element 122 and into a proximal-end portion of the inner tube 112 of the compound tube 104. With a slip- or lock-type fitting, the Luer connector 124 is configured to fluidly connect the lumen of the compound tube 104 to another medical device such a syringe for aspiration or injection of a contrast medium as set forth below.

Each element of the compound hub 106 including at least the housing 120, the rotatable element 122, and the Luer connector 124 can be formed of a polymeric material such as polycarbonate or polypropylene.

The articulation mechanism 108 is a continuous-step articulation mechanism (as opposed to a stepwise articulation mechanism with discrete steps) formed between different elements of the compound tube 104, the cannula tip 110, and the compound hub 106 and controlled by at least rotating the rotatable element 122.

Each tube of the inner tube 112 and the outer tube 114 of the compound tube 104 is fixedly attached to a remainder of the steerable cannula 100 in two places for a total of four places; however, one place of the foregoing places to which the inner tube 112 is attached is movable, which allows the inner tube 112 to be longitudinally moved relative to the outer tube 114 when the rotatable element 122 of the compound hub 106 is rotated in the first direction or the second direction to adjust the cannula angle. Specifically, a distal end of each tube of the inner tube 112 and the outer tube 114 is attached to the cannula tip 110, while a proximal end of the outer tube 114 is attached to a stationary element 130 (e.g., a block) disposed in the housing 120 of the compound hub 106. The inner tube 112 passes through the stationary element 130 to the threaded plug 128, to which a proximal end of the inner tube 112 is attached such as by a pin through the inner tube 112 and the threaded plug 128.

The rotatable element 122 of the compound hub 106 is configured to rotate relative to the housing 120 of the compound hub 106, which drives the threaded plug 128 proximally when the rotatable element 122 is rotated in the first direction or distally when the rotatable element 122 is rotated in the second direction. Because the inner tube 112 of the compound tube 104 is attached to the threaded plug 128, the proximal end of the inner tube 112 is proximally or distally driven in concert with the threaded plug 128. Tension is created in the inner tube 112 when the threaded plug 128 is proximally driven, thereby causing the compound tube 104 to deflect at the flexible hinge 102 with a corresponding cannula angle. Tension is released in the inner tube 112 when the threaded plug 128 is subsequently distally driven, thereby causing the compound tube 104 to relax at the flexible hinge 102 diminishing the foregoing cannula angle.

As such, the steerable cannula 100 is configured for at least a TIPS procedure. The steerable cannula 100 supports anatomical variations in patients, different modes of access within the liver, or both the anatomical variations and the different modes of access. With respect to the support for the anatomical variations or the different modes of access, the cannula angle of the steerable cannula 100 can be adjusted as set forth above to any angle between about −90° and 90°, including about 0° and 90°, such as about 0° and 45°, for example, about 0° and 35° to accommodate the anatomical variations or the different modes of access. With further respect to the different modes of access, the steerable cannula 100 is configured to stiffen a catheter assembly in which it is used in support of a piercing-device throw (e.g., a needle throw, trocar stylet throw, etc.) from the steerable cannula 100 at a chosen cannula angle for a particular mode of access. For example, the steerable cannula 100 stiffens a catheter assembly in a hepatic vein for an anterior needle throw from a right hepatic vein to a right portal vein, an anterior or posterior needle throw from a middle hepatic vein to a left portal vein or the right portal vein, or a posterior needle throw from a left hepatic vein to the left portal vein, in accordance with the chosen cannula angle for the particular mode of access.

### Hinged Needles

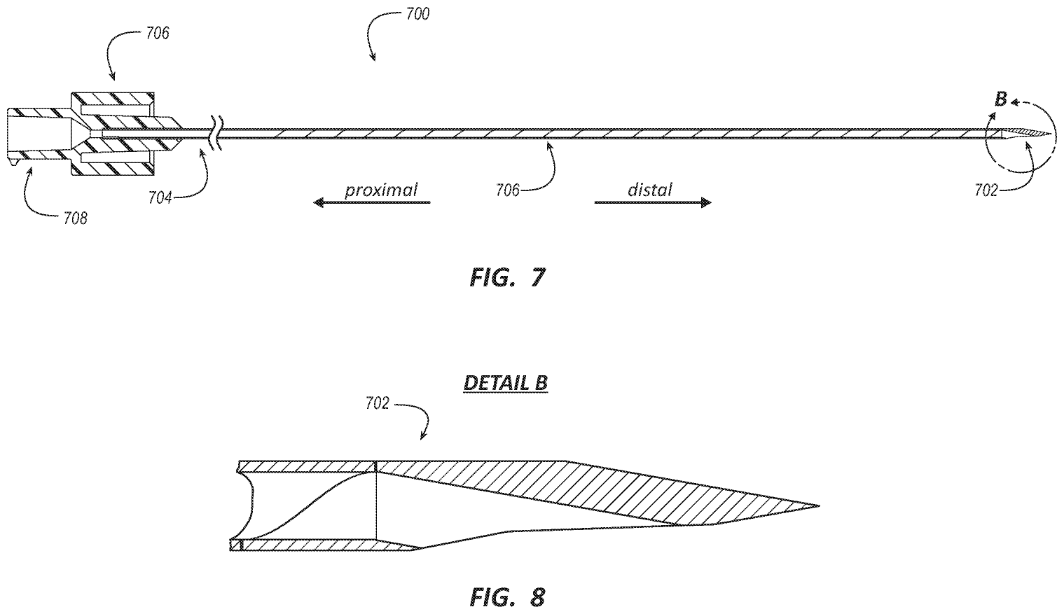

FIG. 7 illustrates a cross section of a hinged needle 700 in accordance with some embodiments. FIG. 8 illustrates a cross section of a needle tip 702 of the hinged needle 700 in accordance with some embodiments.

The hinged needle 700 includes, in some embodiments, an elongate needle tube 704, the needle tip 702, and a hub 706. Each component of the needle tube 704, the needle tip 702, and the hub 706 is described in turn below.

The needle tube 704 includes a lumen extending through an entirety of the needle tube 704, as well as the flexible hinge 706 in a distal-end portion of the needle tube 704.

The lumen of the needle tube 704 is configured for fluid communication between distal and proximal ends of the hinged needle 700. For example, the hinged needle 700 is configured to provide instant blood flashback when the needle tip 702 is properly placed in at least a branch of a portal vein. The hinged needle 700 is also configured to convey a contrast medium into the foregoing branch of the portal vein upon confirming the blood flashback. The lumen of the needle tube 704 is best seen in FIG. 7, which includes a cross section of the needle tube 704.

The flexible hinge 706 of the needle tube 704 is configured to curve when the hinged needle 700 is inserted into a curved channel such as that of the lumen of the steerable cannula 100 when the flexible hinge 102 is adjusted to some cannula angle. Because the cannula angle of the steerable cannula 100 can be adjusted to any angle between about −90° and 90°, including about 0° and 90°, such as about 0° and 45°, for example, about 0° and 35° for a needle throw with a needle such as the hinged needle 700, the flexible hinge 706 is likewise configured to curve with a needle angle between proximal and distal portions of the needle tube 704 adjacent the flexible hinge 706 in accordance with any cannula-tube angle of the foregoing cannula-tube angles.

An entirety of the needle tube 704 or just a portion of the needle tube 704 can be a metal hypotube such as a stainless steel hypotube or a nitinol hypotube. For example, an entirety of the needle tube 704 can be a stainless steel or nitinol hypotube, wherein the flexible hinge 706 is the distal-end portion of such a needle tube. Alternatively, an entirety of the needle tube 704 can be a stainless steel hypotube excepting the distal-end portion of the needle tube 704 including the flexible hinge 706, which can be a nitinol hypotube coupled to the stainless steel hypotube. In another example, an entirety of the needle tube 704 can be a stainless steel or nitinol hypotube, wherein the flexible hinge 706 is a spiral-cut or slotted portion of the hypotube. In yet another example, an entirety of the needle tube 704 can be a stainless steel or nitinol hypotube excepting the distal-end portion of the needle tube 704 including the flexible hinge 706, which can be a spring or braided tube coupled to the hypotube. Optionally, at least a portion of the needle tube 704 such as the distal-end portion of the needle tube 704 including the flexible hinge 706 has a polymer coating (e.g., silicone, polytetrafluoroethylene [“PTFE”], etc.) configured to facilitate needle-tract creation.

The needle tip 702 (e.g., a beveled needle tip, a pencil-point needle tip, etc.) is coupled to a distal end of the needle tube 704 or flexible hinge 706 configured to pierce at least a liver parenchyma and a branch of a portal vein. The needle tip 702 includes a tip opening fluidly connected to the lumen of the needle tube 704. The tip opening of the needle tip 702 is distal to where the needle tip 702 is coupled to the distal end of the needle tube 704 but proximal of a distal end of the needle tip 702. The tip opening in the needle tip 702 is best seen in FIG. 8, which includes a cross section of the needle tip 702.

The hub 706 is disposed about a proximal-end portion of the needle tube 704. The hub 706 includes a Luer connector 708 having an opening corresponding to a hub opening fluidly connected to the lumen of the needle tube 704.

The Luer connector 708 is a female Luer connector extending through the hub 706 or molded with the hub for disposal around the proximal-end portion of the needle tube 704. With a slip- or lock-type fitting, the Luer connector 708 is configured to fluidly connect the lumen of the needle tube 704 to another medical device such a syringe for aspiration or injection of a contrast medium as set forth below.

As such, the hinged needle 700 is configured for at least a TIPS procedure. The hinged needle 700 supports anatomical variations in patients, different modes of access within the liver, or both the anatomical variations and the different modes of access. With respect to the support for the anatomical variations or the different modes of access, the needle angle of the hinged needle 700 can be in accordance with any angle between about −90° and 90°, including about 0° and 90°, such as about 0° and 45°, for example, about 0° and 35° to accommodate the anatomical variations or the different modes of access. With further respect to the different modes of access, the steerable cannula 100 is configured to stiffen a catheter assembly in support of a needle throw from the steerable cannula 100 at a chosen cannula angle for a particular mode of access as set forth above. The hinged needle 700 can accommodate any cannula-tube angle of the foregoing cannula-tube angles for an anterior needle throw from a right hepatic vein to a right portal vein, an anterior or posterior needle throw from a middle hepatic vein to a left portal vein or the right portal vein, or a posterior needle throw from a left hepatic vein to the left portal vein, in accordance with the chosen cannula angle for the particular mode of access.

### Steerable Needles

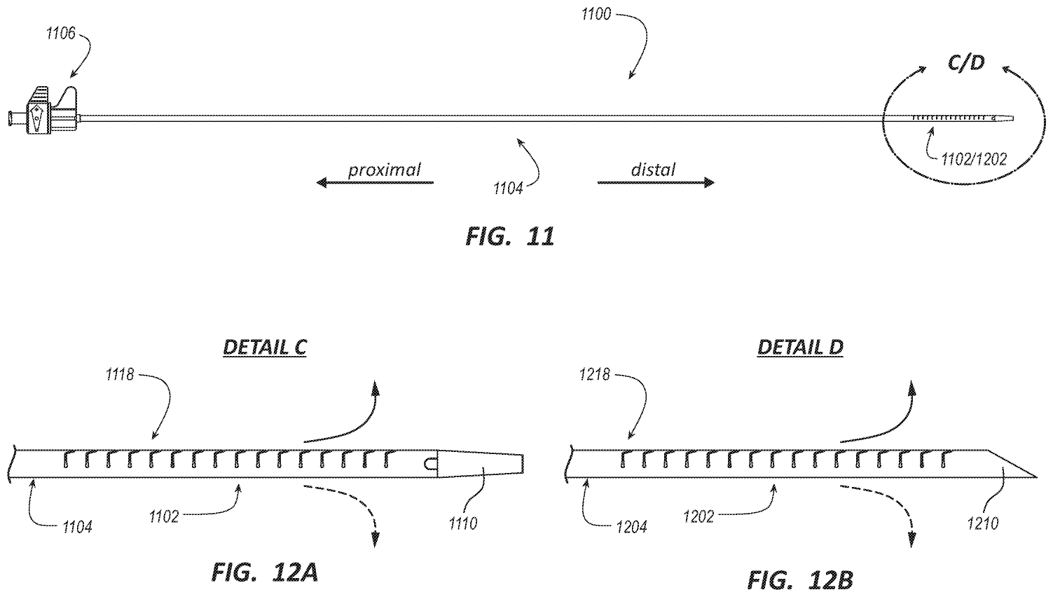

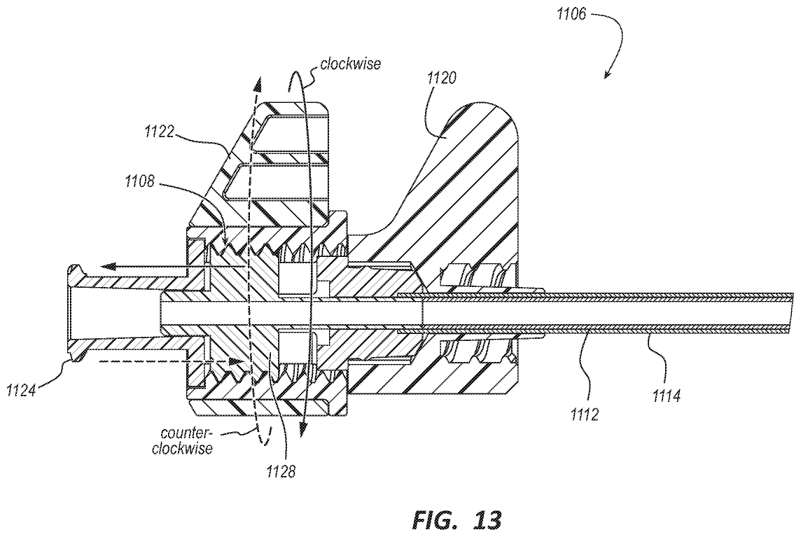

FIG. 11 illustrates a steerable needle 1100 in accordance with some embodiments. FIG. 12A illustrates a flexible hinge 1102 of an elongate compound tube 1104 of the steerable needle 1100 in accordance with some embodiments. FIG. 12B illustrates the flexible hinge 1102 of the compound tube 1104 of the steerable needle 1100 having an alternative needle tip in accordance with some embodiments. FIG. 13 illustrates an articulation mechanism 1108 of the steerable needle 1100 in accordance with some embodiments.

The steerable needle 1100 includes, in some embodiments, the compound tube 1104, a needle tip 1110, a compound hub 1106, and the articulation mechanism 1108. Each component of the compound tube 1104, the needle tip 1110, the compound hub 1106, and the articulation mechanism 1108 is described in turn below. Due to the articulation mechanism 1108 being formed between different elements of the compound tube 1104, the needle tip 1110, and the compound hub 1106, some elements of the compound tube 1104, the needle tip 1110, and the compound hub 1106 are described with respect to the articulation mechanism 1108.

The compound tube 1104 includes an inner tube 1112, an outer tube 1114, a lumen extending through an entirety of the compound tube 1104, and the flexible hinge 1102 in a distal-end portion of the compound tube 1104.

The lumen of the compound tube 1104 is configured for fluid communication between distal and proximal ends of the steerable needle 1100. For example, the steerable needle 1100 is configured to provide instant blood flashback when the needle tip 1110 is properly placed in at least a branch of a portal vein. The steerable needle 1100 is also configured to convey a contrast medium into the foregoing branch of the portal vein upon confirming the blood flashback. The lumen of the compound tube 1104 is best seen in FIG. 13, which includes a cross section of the compound tube 1104.

The flexible hinge 1102 of the compound tube 1104 is configured for adjusting a needle angle between proximal and distal portions of the compound tube 1104 adjacent the flexible hinge 1102 in support of a needle throw from a catheter assembly including the steerable needle 1100. The needle angle of the steerable needle 1100 can be adjusted to any angle between about −90° and 90°, including about 0° and 90°, such as about 0° and 45°, for example, about 0° and 35° for such a needle throw.

The flexible hinge 1102 of the compound tube 1104 is formed between offset sets of transverse cutouts 1116 (not shown) and 1118 in at least a non-deflected state of the compound tube 1104. Each tube of the inner tube 1112 and the outer tube 1114 of the compound tube 1104 includes a set of the cutouts, respectively the set of cutouts 1116 and the set of cutouts 1118. The set of cutouts 1116 in the outer tube 1114 is best seen in FIG. 12A. Each set of the sets of cutouts 1116 and 1118 can be laser cutouts. The laser cutouts are precise cutouts characterized by having edges with high-quality surface finishes.

An entirety of the compound tube 1104 (e.g., an entirety of both the inner tube 1112 and the outer tube 1114) or just a portion of the compound tube 1104 (e.g., the inner tube 1112, the outer tube 1114, or a portion of any one or both of the foregoing tubes) can be a metal hypotube such as a stainless steel hypotube or a nitinol hypotube. For example, an entirety of the compound tube 1104 can be a stainless steel or nitinol hypotube, wherein the flexible hinge 1102 is a spiral-cut or slotted portion of the hypotube. That is, each set of cutouts of the set of cutouts 1116 of the inner tube 1112 and the set of cutouts 1118 of the outer tube 1114 is a spiral-cut or slotted portion of the hypotube respectively forming the inner tube 1112 and the outer tube 1114 of the compound tube 1104. Optionally, at least a portion of the compound tube 1104 such as the distal-end portion of the compound tube 1104 including the flexible hinge 1102 has a polymer coating (e.g., silicone, PTFE, etc.) configured to facilitate needle-tract creation.

The needle tip 1110 is coupled to a distal end of the compound tube 1104 or flexible hinge 1102 configured to pierce at least a liver parenchyma and a branch of a portal vein. A distal end of each tube of the inner tube 1112 and the outer tube 1114 of the compound tube 1104 is fixedly attached to the needle tip 1110. The needle tip 1110 includes a tip opening fluidly connected to the lumen of the compound tube 1104. As shown in FIG. 12A, the needle tip 1110 can be a blunt needle tip having a tip opening in a distal end of the needle tip 1110. However, like the needle tip 702 of the hinged needle 700, the needle tip 1110 can be a beveled needle tip or a pencil-point needle tip having the tip opening of the needle tip 1110 distal to where the needle tip 1110 is coupled to the distal end of the compound tube 1104 but proximal of a distal end of the needle tip 1110.

The compound hub 1106 includes a fixed element 1120 (e.g., a fixed handle), a rotatable element 1122 (e.g., a rotatable handle), and a Luer connector 1124. The compound hub 1106 is disposed about a proximal-end portion of the compound tube 1104.

The fixed element 1120 of the compound hub 1106 can be formed of a unitary molded piece over the proximal-end portion of the compound tube 1104. FIG. 13 illustrates such a molded piece in cross section.

The rotatable element 1122 of the compound hub 1106 is configured to rotate relative to the fixed element 1120 of the compound hub 1106. As best shown in FIG. 13, the rotatable element 1122 includes a threaded bore having internal threads configured to drive a threaded plug 1128 having external threads proximally when the rotatable element 1122 is rotated in the first direction or distally when the rotatable element 1122 is rotated in the second direction.

The Luer connector 1124 is a female Luer connector disposed in the rotatable element 1122 over a tubular extension of a distal-end portion of the threaded plug 1128. With a slip- or lock-type fitting, the Luer connector 1124 is configured to fluidly connect the lumen of the compound tube 1104 to another medical device such a syringe for aspiration or injection of a contrast medium as set forth below.

Each element of the compound hub 1106 including at least the fixed element 1120, the rotatable element 1122, and the Luer connector 1124 can be formed of a polymeric material such as polycarbonate or polypropylene.

The articulation mechanism 1108 is a continuous-step articulation mechanism (as opposed to a stepwise articulation mechanism with discrete steps) formed between different elements of the compound tube 1104, the needle tip 1110, and the compound hub 1106 and controlled by at least rotating the rotatable element 1122.

Each tube of the inner tube 1112 and the outer tube 1114 of the compound tube 1104 is fixedly attached to a remainder of the steerable needle 1100 in two places for a total of four places; however, one place of the foregoing places to which the inner tube 1112 is attached is movable, which allows the inner tube 1112 to be longitudinally moved relative to the outer tube 1114 when the rotatable element 1122 of the compound hub 1106 is rotated in the first direction or the second direction to adjust the needle angle. Specifically, a distal end of each tube of the inner tube 1112 and the outer tube 1114 is attached to the needle tip 1110, while a proximal-end portion of the outer tube 1114 is attached to the fixed element 1120. The inner tube 1112 passes through the fixed element 112 to the threaded plug 1128, to which a proximal end of the inner tube 1112 is attached.

The rotatable element 1122 of the compound hub 1106 is configured to rotate relative to the fixed element 1120 of the compound hub 1106, which drives the threaded plug 1128 proximally when the rotatable element 1122 is rotated in the first direction or distally when the rotatable element 1122 is rotated in the second direction. Because the inner tube 1112 of the compound tube 1104 is attached to the threaded plug 1128, the proximal end of the inner tube 1112 is proximally or distally driven in concert with the threaded plug 1128. Tension is created in the inner tube 1112 when the threaded plug 1128 is proximally driven, thereby causing the compound tube 1104 to deflect at the flexible hinge 1102 with a corresponding needle angle. Tension is released in the inner tube 1112 when the threaded plug 1128 is subsequently distally driven, thereby causing the compound tube 1104 to relax at the flexible hinge 1102 diminishing the foregoing needle angle.

In an alternative embodiment to the steerable needle 1100 set forth above, the steerable needle 110 can include a single elongate tube 1204 like the inner tube 1112 or the outer tube 1114 instead of the compound tube 1104, a wire-driven articulation mechanism instead of a tube-driven articulation mechanism like the articulation mechanism 1108, and a beveled needle tip 1210 instead of the needle tip 1110 as shown in FIG. 12B. Other elements of the alternative embodiment to the steerable needle 1100 can be largely similar if not identical but for features needed by the wire-driven articulation mechanism.

The wire-driven articulation mechanism of the alternative embodiment to the steerable needle 1100 includes one or two tensioned control lines extending from the threaded plug 1128, through the single tube 1204 beyond a flexible hinge 1202 having a set of cutouts 1218 like the flexible hinge 1102, and to a distal-end portion of the single tube 1204 for adjusting the needle angle. The rotatable element 1122 of the compound hub 1106 of the alternative embodiment to the steerable needle 1100 is configured to rotate relative to the fixed element 1120 of the compound hub 1106, which drives the threaded plug 1128 proximally when the rotatable element 1122 is rotated in the first direction or distally when the rotatable element 1122 is rotated in the second direction. Because the one or two control lines are attached to the threaded plug 1128, the one or two control lines are proximally or distally driven in concert with the threaded plug 1128. Tension is created in the single tube 1204 when the threaded plug 1128 is proximally driven, thereby causing the single tube 1204 to deflect at the flexible hinge 1202 with a corresponding needle angle (e.g., about −90° to about 90°). Tension is released in the single tube 1204 when the threaded plug 1128 is subsequently distally driven, thereby causing the single tube 1204 to relax at the flexible hinge 1202 diminishing the foregoing needle angle.

As such, the steerable needle 1100 or the alternative embodiment thereof is configured for at least a TIPS procedure. The steerable needle 1100 or the alternative embodiment thereof supports anatomical variations in patients, different modes of access within the liver, or both the anatomical variations and the different modes of access. With respect to the support for the anatomical variations or the different modes of access, the needle angle of the steerable needle 1100 or the alternative embodiment thereof can be adjusted as set forth above to any angle between about −90° and 90°, including about 0° and 90°, such as about 0° and 45°, for example, about 0° and 35° to accommodate the anatomical variations or the different modes of access. With further respect to the different modes of access, the steerable needle 1100 or the alternative embodiment thereof is configured with sufficient stiffness for a needle throw from a catheter assembly without a stiffening cannula at a chosen needle angle for a particular mode of access. For example, the steerable needle 1100 or the alternative embodiment thereof, itself, stiffens the catheter assembly in a hepatic vein enabling an anterior needle throw from a right hepatic vein to a right portal vein, an anterior or posterior needle throw from a middle hepatic vein to a left portal vein or the right portal vein, or a posterior needle throw from a left hepatic vein to the left portal vein, in accordance with the chosen needle angle for the particular mode of access.

### Access Sets

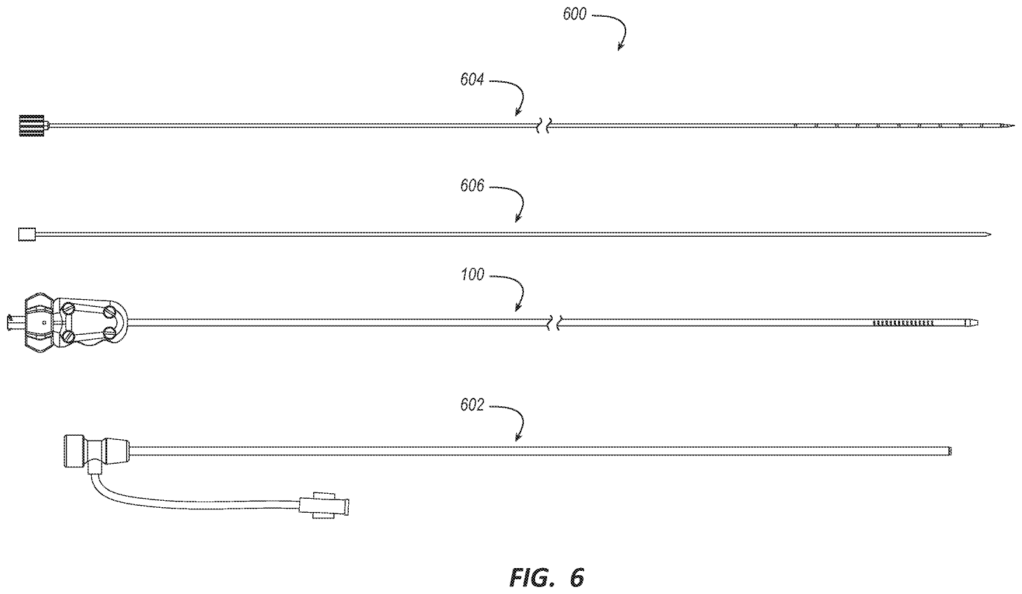

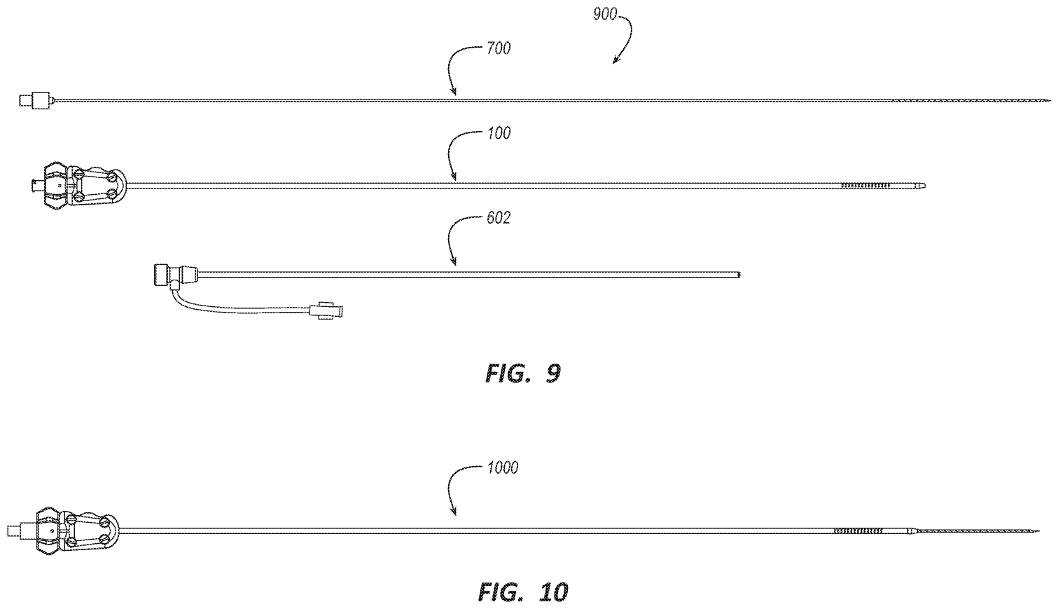

FIG. 6 illustrates an access set 600 including the steerable cannula 100 in accordance with some embodiments. FIG. 9 illustrates an access set 900 including the hinged needle 700 and the steerable cannula 100 in accordance with some embodiments. FIG. 10 illustrates an assembly 1000 of the hinged needle 700 disposed in the steerable cannula 100 in accordance with some embodiments.

The access set 600 or 900 includes, in some embodiments, an introducer sheath 602, a stiffening cannula, and a piercing device. Each component of the introducer sheath 602, the stiffening cannula, and the piercing device is described in turn below.

The introducer sheath 602 is configured to hold a blood vessel, piercing-device tract, or the like open during a percutaneous procedure such as a TIPS procedure for at least introduction of other components of the access set 600 or 900 during the percutaneous procedure. The introducer sheath 602 includes a primary tube, a primary hub, and a primary lumen longitudinally extending through the primary tube and the primary hub of the introducer sheath 602. The introducer sheath 602 also includes a secondary tube, a secondary hub, and a secondary lumen extending through the secondary tube and the secondary hub of the introducer sheath 602. The foregoing secondary elements of the introducer sheath 602 can also be respectively referred to as a side tube, a side-tube hub, and a side-tube lumen.

While not shown, the access set 600 or 900 can further include a dilator for combination with the introducer sheath 602 in a first assembly. The introducer sheath 602 is configured to accept the dilator in the primary lumen of the introducer sheath 602 such that a distal-end portion of the dilator extends past a distal end of the introducer sheath 602 for dilation of an access site, blood vessel, piercing-device tract, or the like during a percutaneous procedure.

The stiffening cannula is selected from the steerable cannula 100 and a fixed-curve cannula, each of which is configured to support a piercing-device throw therefrom.

The piercing device is selected from the hinged needle 700, a Colapinto needle, and a trocar stylet 604.

Preferably, the access set 600 or 900 includes at least the steerable cannula 100 or the hinged needle 700, each of which is set forth above in detail.

While not shown, the access set 600 or 900 can further include an access device, one or more catheters, or any of a number of other components configured to facilitate a percutaneous procedure such as a TIPS procedure. Examples of such components are described in turn below with the understanding that the access set 600 or 900 can further include each component of the access device and the one or more catheters independently of any other component of the dilator, the access device, and the one or more catheters.

The access set 600 or 900 can further include the access device and a first catheter of the one or more catheters. The access device is configured to create an access site for a percutaneous procedure such as an access site in a jugular vein for a TIPS procedure. The first catheter is configured to catheterize a blood vessel from the access site such as an inferior vena cava from the access site in the jugular vein.

The access set 600 or 900 can further include a second catheter of the one or more catheters for combination with the stiffening cannula (e.g., the steerable cannula 100) in a second assembly. The second catheter is configured to accept the stiffening cannula in a lumen of the second catheter and conceal a distal end of the stiffening cannula with a distal-end portion of the second catheter. This protects other components of the access set 600 or 900 from being damaged by the distal end of the stiffening cannula when advancing the second assembly in vivo. If the stiffening cannula is the steerable cannula 100, the second catheter can, if needed, also prevent damage to components of the access set 600 or 900 when articulating the stiffening cannula 100.

The access set 600 or 900 can further include a third catheter (e.g., catheter 606) of the one or more catheters for combination with the piercing device (e.g., the hinged needle 700) in a third assembly. The third catheter is configured to accept the piercing device in a lumen of the third catheter and conceal a distal end of the piercing device with a distal-end portion of the third catheter. This protects other components of the access set 600 or 900 from being pierced by the distal end of the piercing device when advancing the third assembly in vivo. If the piercing device is the hinged needle 700, the third catheter can, if needed, also prevent damage to components of the access set 600 or 900 when the hinged needle 700 is curved to accommodate a curved channel.

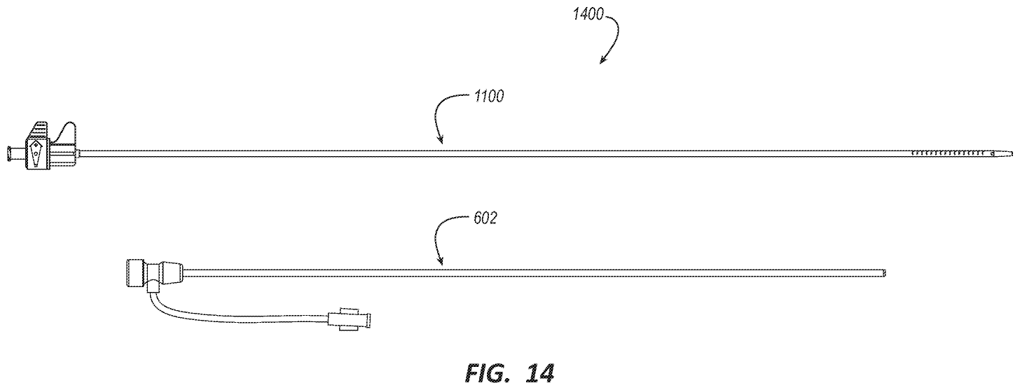

FIG. 14 illustrates an access set 1400 including the steerable needle 1100 in accordance with some embodiments.

The access set 1400 includes, in some embodiments, the introducer sheath 602 and the steerable needle 1100, each of which is set forth above in detail.

While not shown, the access set 1400 can further include the dilator set forth above for combination with the introducer sheath 602 in the first assembly.

While not shown, the access set 1400 can further include an access device, one or more catheters, or any of a number of other components configured to facilitate a percutaneous procedure such as a TIPS procedure. Examples of such components are described in turn below with the understanding that the access set 1400 can further include each component of the access device and the one or more catheters independently of any other component of the dilator, the access device, and the one or more catheters.

The access set 1400 can further include the access device and a first catheter of the one or more catheters. The access device is configured to create an access site for a percutaneous procedure such as an access site in a jugular vein for a TIPS procedure. The first catheter is configured to catheterize a blood vessel from the access site such as an inferior vena cava from the access site in the jugular vein.

The access set 1400 can further include a third catheter (e.g., the catheter 606 of FIG. 6) of the one or more catheters for combination with the steerable needle 1100 in a third assembly. Like the third catheter of the access set 600 or 900, when present, the third catheter of the access set 1400 is configured to accept the steerable needle 1100 in a lumen of the third catheter and conceal a distal end of the steerable needle 1100 with a distal-end portion of the third catheter. This protects other components of the access set 1400 from being pierced by the distal end of the steerable needle 1100 when advancing the third assembly in vivo. The third catheter can, if needed, also prevent damage to components of the access set 1400 when the steerable needle 1100 is articulated.

### Methods

FIG. 15 illustrates a percutaneous procedure involving placement of a TIPS between a hepatic vein and a portal vein in accordance with some embodiments.

Methods of steerable cannulas, hinged or steerable needles, or access sets thereof include methods of using the steerable cannula 100, the hinged needle 700, the steerable needle 1100, or the access set 600, 900, or 1400 in percutaneous procedures such as TIPS procedures. For consistency, components of the access sets 600, 900, and 1400 using ordinal numbers such as the first catheter, the second catheter, and the third catheter are intended to be the same components set forth in the methods below.

A method for a TIPS procedure using a hollow needle such as the hinged needle 700 can include any one or more steps of a number of steps set forth below.

The method can include creating an access site in a right jugular vein with an access device (e.g., an access needle) and subsequently advancing an access guidewire into an inferior vena cava. The access device can be optionally removed, but the access guidewire should be left in place in the inferior vena cava.

The method can include advancing a first catheter over the access guidewire to the inferior vena cava, removing the access guidewire, and advancing a first maneuver guidewire to the inferior vena cava. A right hepatic vein or a most adequate alternative branch (e.g., either a middle or a left hepatic vein) can be selected with the first maneuver guidewire and subsequently catheterized with the first catheter. The first catheter can be removed, but the first maneuver guidewire should be left in place as distal as safely possible in the right hepatic vein or alternative branch.

The method can include dilating the access site with a first dilator to form a dilated access site. A first assembly of a second dilator in the introducer sheath 602 can be advanced through the dilated access site over the first maneuver guidewire as distal as safely possible into the right hepatic vein or alternative branch. The first maneuver guidewire and the second dilator can be removed, but the introducer sheath 602 should be left in place in the right hepatic vein or alternative branch.

The method can include advancing a second assembly of the hinged needle 700 in a second catheter into the right hepatic vein or alternative branch. A tip of the hinged needle 700 should not extend beyond a tip of the second catheter.

The method can include orienting a tip of the second assembly (i.e., the tip of the second catheter) toward a right portal vein or an adequate alternative branch (e.g., a either a middle or left portal vein), wedging the tip of the second assembly against a wall of the right hepatic vein or alternative branch, and thrusting the hinged needle 700 through the tip of the second catheter, through the wall of the right hepatic vein or alternative branch, through a liver parenchyma, and into the right portal vein or alternative branch.

The method can include connecting a tip of a syringe having a barrel optionally filled with a contrast medium to the hub 706 of the hinged needle 700, pulling a plunger of the syringe to create a vacuum, and slowly withdrawing the hinged needle 700 until blood is seen in the barrel of the syringe, thereby confirming the tip of the hinged needle 700 is in the right portal vein or alternative branch.

The method can include injecting the contrast medium and subsequently radiographically confirming the tip of the hinged needle 700 is in the right portal vein or alternative branch.

The method can include advancing the second catheter over the hinged needle 700 into the right portal vein or alternative branch. The hinged needle 700 can be removed, but the second catheter should be left in place in the right portal vein or alternative branch.

The method can include advancing a second maneuver guidewire into the right portal vein or alternative branch, selecting a main portal vein, and advancing the second catheter and the introducer sheath 602 over the second maneuver guidewire across the liver parenchyma, through the right portal vein or alternate branch, and into a main portal vein.

The method can include proceeding with any indicated interventional procedures such as dilating the tract across the liver parenchyma with a balloon and placing a TIPS as shown in FIG. 15.

In view of the foregoing method, another method for a TIPS procedure using the hinged needle 700 includes, in some embodiments, a first cannula-inserting step, a first needle-inserting step, a cannula-orienting step, a needle-throwing step, and, optionally, any one or more steps or portions thereof provided in the foregoing method for a TIPS procedure using a hollow needle.

The first cannula-inserting step includes inserting a stiffening cannula into the introducer sheath 602 positioned in a distal portion of a hepatic vein. The stiffening cannula is configured to support a needle throw from the stiffening cannula.

The first needle-inserting step includes inserting the hinged needle 700 into the stiffening cannula.

The cannula-orienting step includes orienting the stiffening cannula toward a portal vein.

The needle-throwing step includes throwing the hinged needle 700 from the hepatic vein to the portal vein in accordance with an orientation of the stiffening cannula. The needle-throwing step includes throwing the hinged needle from a right hepatic vein to a right portal vein with an anterior needle throw, a middle hepatic vein to either a left portal vein or the right portal vein respectively with either a different anterior needle throw or a posterior needle throw, or a left hepatic vein to the left portal vein with a different posterior needle throw. The needle-throwing step is in accordance with the orientation of the stiffening cannula.

The method can further include a syringe-connecting step, a plunger-pulling step, and a portal vein-confirming step. The syringe-connecting step includes connecting a tip of a syringe having a barrel optionally filled with a contrast medium to a hub of the hinged needle 700. The plunger-pulling step includes pulling a plunger of the syringe to create a vacuum. The portal vein-confirming step includes slowly withdrawing the hinged needle 700 until blood is seen in the barrel of the syringe. The portal vein-confirming step confirms the needle tip of the hinged needle 700 is in the portal vein.

The method can further include a second cannula-inserting step. The second cannula-inserting step includes inserting the stiffening cannula into a second catheter to form a second catheter assembly before inserting the stiffening cannula into the introducer sheath 602. The stiffening cannula is inserted into the introducer sheath 602 as part of the second catheter assembly when the second cannula-inserting step is performed.

The method can further include a second needle-inserting step. The second needle-inserting step includes inserting the hinged needle 700 into a third catheter to form a third catheter assembly before inserting the hinged needle 700 into the stiffening cannula. The hinged needle 700 is inserted into the stiffening cannula as part of the third catheter assembly when the second needle-inserting step is performed.

Another method for a TIPS procedure using a solid needle such as the trocar stylet 604 can include any one or more steps of a number of steps set forth below.

The method can include creating an access site in a right jugular vein with an access device (e.g., an access needle) and subsequently advancing an access guidewire into an inferior vena cava. The access device can be optionally removed, but the access guidewire should be left in place in the inferior vena cava.

The method can include advancing a first catheter over the access guidewire to the inferior vena cava, removing the access guidewire, and advancing a first maneuver guidewire to the inferior vena cava. A right hepatic vein or a most adequate alternative branch (e.g., either a middle or a left hepatic vein) can be selected with the first maneuver guidewire and subsequently catheterized with the first catheter. The first catheter can be removed, but the first maneuver guidewire should be left in place as distal as safely possible in the right hepatic vein or alternative branch.

The method can include dilating the access site with a first dilator to form a dilated access site. A first assembly of a second dilator in the introducer sheath 602 can be advanced through the dilated access site over the first maneuver guidewire as distal as safely possible into the right hepatic vein or alternative branch. The second dilator can be removed, but the first maneuver guidewire and the introducer sheath 602 should be left in place in the right hepatic vein or alternative branch.

The method can include advancing a second assembly of a stiffening cannula in a second catheter into the right hepatic vein or alternative branch. The first maneuver guidewire can be removed, but the second assembly should be left in place in the right hepatic vein or alternative branch.

The method can include advancing a third assembly of the trocar stylet 604 in a third catheter into the right hepatic vein or alternative branch. A tip of the trocar stylet 604 should not extend beyond a tip of the third catheter.

The method can include orienting a tip of the second assembly (e.g., a tip of the second catheter) toward a right portal vein or an adequate alternative branch (e.g., a either a middle or left portal vein), wedging the tip of the second assembly against a wall of the right hepatic vein or alternative branch, and thrusting the third assembly led by the tip of the trocar stylet 604 through the tip of the second assembly, through the wall of the right hepatic vein or alternative branch, through a liver parenchyma, and into the right portal vein or alternative branch. The trocar stylet 604 can be removed, but the third catheter should be left in place in the right portal vein or alternative branch.

The method can include connecting a tip of a syringe having a barrel optionally filled with a contrast medium to a hub of the third catheter, pulling a plunger of the syringe to create a vacuum, and slowly withdrawing the third catheter until blood is seen in the barrel of the syringe, thereby confirming the tip of the third catheter is in the right portal vein or alternative branch.

The method can include injecting the contrast medium and subsequently radiographically confirming the tip of the third catheter is in the right portal vein or alternative branch.

The method can include advancing a second maneuver guidewire into the right portal vein or alternative branch, selecting a main portal vein, and, while holding the stiffening cannula in position, advancing the second catheter and the introducer sheath 602 over the third catheter and the second maneuver guidewire across the liver parenchyma, through the right portal vein or alternate branch, and into the main portal vein. The third catheter and the stiffening cannula can be removed, but the second maneuver guidewire, the second catheter, and the introducer sheath 602 should be left in place in the main portal vein.

The method procedure can include proceeding with any indicated interventional procedures such as dilating the tract across the liver parenchyma with a balloon and placing a TIPS as shown in FIG. 15.

In view of any method of the foregoing methods, another method for a TIPS procedure using the steerable cannula 100 includes, in some embodiments, a first cannula-inserting step, a first piercing device-inserting step, a cannula-articulating step, a cannula angle-choosing step, a piercing device-throwing step, and, optionally, any one or more steps or portions thereof provided in the foregoing methods for TIPS procedures.

The first cannula-inserting step includes inserting the steerable cannula 100 into the introducer sheath 602 positioned in a distal portion of a hepatic vein.

The first piercing device-inserting step includes inserting a piercing device into the steerable cannula 100.

The cannula-articulating step includes articulating the steerable cannula 100 by rotating the rotatable element 122 of the compound hub 106 of the steerable cannula 100 either before or after inserting the piercing device into the steerable cannula 100.

The cannula angle-choosing step includes choosing the cannula angle for the piercing-device throw from the hepatic vein to a portal vein.

The piercing device-throwing step includes throwing the piercing device from the hepatic vein to the portal vein in accordance with the cannula angle and with support provided by the compound tube 104 of the steerable cannula 100. The piercing device-throwing step includes throwing the piercing device from a right hepatic vein to a right portal vein with an anterior piercing-device throw, a middle hepatic vein to either a left portal vein or the right portal vein respectively with either a different anterior piercing-device throw or a posterior piercing-device throw, or a left hepatic vein to the left portal vein with a different posterior piercing-device throw. The piercing device-throwing step is in accordance with the cannula angle chosen for the piercing-device throw.

The method further can further include a second cannula-inserting step. The second cannula-inserting step includes inserting the steerable cannula 100 into a second catheter to form a second catheter assembly before inserting the steerable cannula 100 into the introducer sheath 602. The steerable cannula 100 is inserted into the introducer sheath 602 as part of the second catheter assembly when the second cannula-inserting step is performed.

The method further can further include a second piercing device-inserting step. The second piercing device-inserting step includes inserting the piercing device into a third catheter to form a third catheter assembly before inserting the piercing device into the steerable cannula 100. The piercing device is inserted into the steerable cannula 100 as part of the third catheter assembly when the second piercing device-inserting step is performed.

In view of any method of the foregoing methods, another method for a TIPS procedure using the steerable needle 1100 includes, in some embodiments, a first needle-inserting step, a needle-articulating step, a needle angle-choosing step, a needle-throwing step, and, optionally, any one or more steps or portions thereof provided in the foregoing methods for TIPS procedures.

The first needle-inserting step includes inserting the steerable needle 1100 into the introducer sheath 602 positioned in a distal portion of a hepatic vein.

The needle-articulating step includes articulating the steerable needle 1100 by rotating the rotatable handle 1122 of the compound hub 1106 of the steerable needle 1100 either before or after inserting the steerable needle 1100 into the introducer sheath 602.

The needle angle-choosing step includes choosing the needle angle for a needle throw from the hepatic vein to a portal vein.

The needle-throwing step include throwing the steerable needle 1100 from the hepatic vein to the portal vein in accordance with the needle angle. The needle-throwing step includes throwing the steerable needle 100 from a right hepatic vein to a right portal vein with an anterior needle throw, a middle hepatic vein to either a left portal vein or the right portal vein respectively with either a different anterior needle throw or a posterior needle throw, or a left hepatic vein to the left portal vein with a different posterior needle throw, in accordance with the needle angle.

The method can further include a syringe-connecting step, a plunger-pulling step, and a portal vein-confirming step. The syringe-connecting step includes connecting a tip of a syringe having a barrel optionally filled with a contrast medium to the Luer connector 1124 of the steerable needle 100. The plunger-pulling step includes pulling a plunger of the syringe to create a vacuum. The portal vein-confirming step includes slowly withdrawing the steerable needle 1100 until blood is seen in the barrel of the syringe. The portal vein-confirming step confirms the needle tip 1110 or 1210 of the steerable needle 1100 is in the portal vein.

The method can further include a second needle-inserting step. The second needle-inserting step includes inserting the steerable needle 1100 into a third catheter to form a third catheter assembly before inserting the steerable needle 1100 into the introducer sheath 602. The steerable needle 1100 is inserted into the introducer sheath 602 as part of the third catheter assembly when the second needle-inserting step is performed.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

1. A steerable cannula for a transjugular intrahepatic portosystemic shunt (“TIPS”) procedure, comprising:
an elongate compound tube having a proximal-end portion and a distal-end portion, the distal-end portion including a flexible hinge for adjusting a cannula angle between proximal and distal portions of the compound tube adjacent the flexible hinge;
a cannula tip coupled to a distal end of the compound tube, the cannula tip including a tip opening fluidly connected to a lumen of the compound tube; and
a compound hub disposed about the proximal-end portion of the compound tube; and
an articulation mechanism controlled by at least a rotatable element of the compound hub for adjusting the cannula angle, the articulation mechanism configured to longitudinally move an inner tube of the compound tube relative to an outer tube of the compound tube to adjust the cannula angle.

2. The steerable cannula of claim 1, wherein each tube of the inner tube and the outer tube of the compound tube includes a set of transverse cutouts, the flexible hinge of the compound tube formed between offset sets of the transverse cutouts in at least a non-deflected state of the compound tube.

3. The steerable cannula of claim 1, wherein the rotatable element of the compound hub is a rotatable handle configured to rotate relative to a housing of the compound hub, the rotatable handle including a threaded bore or an insert including a threaded bore configured to drive a threaded plug proximally when the rotatable handle is rotated in a first direction or distally when the rotatable handle is rotated in a second direction.

4. The steerable cannula of claim 3, wherein the threaded plug is fixedly attached to the inner tube of the compound tube such that the inner tube is driven proximally or distally in concert with the threaded plug when the rotatable handle is respectively rotated in the first direction or the second direction.

5. The steerable cannula of claim 3, wherein the outer tube of the compound tube is fixedly attached to a stationary element disposed in the housing of the compound hub, the inner tube of the compound tube passing through the stationary element.

6. The steerable cannula of claim 3, wherein a distal end of each tube of the inner tube and the outer tube of the compound tube is fixedly attached to the cannula tip, thereby allowing the inner tube to be longitudinally moved relative to the outer tube when the rotatable handle of the compound hub is rotated in the first direction or the second direction.

7. The steerable cannula of claim 1, wherein the compound tube includes a needle lumen longitudinally extending through the compound tube configured for disposing a needle or a catheter-and-needle assembly therein.

8. The steerable cannula of claim 1, wherein the cannula angle ranges from about −90° to about 90°.

9. The steerable cannula of claim 1, wherein the compound hub includes a female Luer connector extending through the rotatable element and into a distal-end portion of the inner tube of the compound tube, the Luer connector configured to fluidly connect the lumen of the compound tube to another medical device.

10. The steerable cannula of claim 1, wherein the steerable cannula is configured to stiffen a catheter assembly in support of a needle throw from the catheter assembly at the cannula angle chosen for the needle throw.

11. A hinged needle for a transjugular intrahepatic portosystemic shunt (“TIPS”) procedure, comprising:
an elongate needle tube having a proximal-end portion and a distal-end portion, the distal-end portion including a flexible hinge configured to curve when the hinged needle is inserted into a curved channel;
a pencil-point needle tip coupled to a distal end of the needle tube, the needle tip including a tip opening fluidly connected to a lumen of the needle tube; and
a hub about the proximal-end portion of the needle tube including a hub opening fluidly connected to the lumen of the needle tube.

12. The hinged needle of claim 11, wherein an entirety of the needle tube is a metal hypotube.

13. The hinged needle of claim 12, wherein the flexible hinge is a spiral-cut or slotted portion of the hypotube.

14. The hinged needle of claim 12, wherein at least the flexible hinge of the hypotube is nitinol.

15. The hinged needle of claim 11, wherein the needle tube is a hypotube coupled to a spring as the flexible hinge.

16. The hinged needle of claim 11, wherein the needle tube is a hypotube coupled to a braided tube as the flexible hinge.

17. The hinged needle of claim 11, wherein the tip opening of the needle tip is distal to where the needle tip is coupled to the distal end of the needle tube.

18. The hinged needle of claim 11, wherein the hub includes a female Luer connector in a proximal-end portion of the hub configured for fluidly connecting the lumen of the needle tube to another medical device.

19. The hinged needle of claim 11, wherein the needle tip is configured to pierce a liver parenchyma and a branch of a portal vein.

20. The hinged needle of claim 11, wherein the hinged needle is configured to provide instant blood flashback when the needle tip is properly placed in at least the branch of the portal vein.

21. An access set for a transjugular intrahepatic portosystemic shunt (“TIPS”) procedure, comprising:
an introducer sheath;
a stiffening cannula selected from a steerable cannula and a fixed-curve cannula configured to support a piercing-device throw from the stiffening cannula, the steerable cannula including an articulation mechanism configured to longitudinally move an inner tube of an elongate compound tube relative to an outer tube of the compound tube to adjust a piercing-device angle for the piercing-device throw; and
a piercing device selected from a hinged needle, a Colapinto needle, and a trocar stylet, the hinged needle including an elongate needle tube having a flexible hinge and a pencil-point needle tip coupled to a distal end of the needle tube having a tip opening fluidly connected to a lumen of the needle tube, wherein the access set includes at least the steerable cannula or the hinged needle.

22. The access set of claim 21, wherein each tube of the inner tube and the outer tube of the compound tube of the steerable cannula includes a set of transverse cutouts forming a flexible hinge of the compound tube, each set of transverse cutouts offset from the other in at least a non-deflected state of the compound tube.

23. The access set of claim 22, wherein a rotatable handle of a compound hub of the steerable cannula is configured to rotate relative to a housing of the compound hub, the rotatable handle including a threaded bore or an insert including a threaded bore configured to drive a threaded plug proximally when the rotatable handle is rotated in a first direction or distally when the rotatable handle is rotated in a second direction.

24. The access set of claim 23, wherein the threaded plug is fixedly attached to the inner tube of the compound tube such that the inner tube is driven proximally or distally in concert with the threaded plug when the rotatable handle is respectively rotated in the first direction or the second direction.

25. The access set of claim 23, wherein the outer tube of the compound tube is fixedly attached to a stationary element disposed in the housing of the compound hub, the inner tube of the compound tube passing through the stationary element.

26. The access set of claim 23, wherein a distal end of each tube of the inner tube and the outer tube of the compound tube is fixedly attached to a cannula tip, thereby allowing the inner tube to be longitudinally moved relative to the outer tube when the rotatable handle of the compound hub is rotated in the first direction or the second direction.

27. The access set of claim 21, wherein an entirety of the needle tube of the hinged needle is a metal hypotube, the flexible hinge selected from a spiral-cut portion of the hypotube, a slotted portion of the hypotube, and a nitinol portion of the hypotube.

28. The access set of claim 21, wherein the needle tube of the hinged needle is a hypotube coupled to a spring or braided tube as the flexible hinge.

29. The access set of claim 21, wherein the tip opening of the needle tip of the hinged needle is distal to where the needle tip is coupled to the distal end of the needle tube.

30. The access set of claim 21, further comprising:
an access device configured to create an access site in at least a jugular vein; and
a first catheter configured to catheterize an inferior vena cava from the access site.

31. The access set of claim 21, further comprising a dilator for combination with the introducer sheath in a first assembly, the introducer sheath configured to accept the dilator in a lumen of the introducer sheath such that a distal-end portion of the dilator extends past a distal end of the introducer sheath for dilation of a piercing-device tract.

32. The access set of claim 21, further comprising a second catheter for combination with the stiffening cannula in a second assembly, the second catheter configured to accept the stiffening cannula in a lumen of the second catheter and conceal a distal end of the stiffening cannula with a distal-end portion of the second catheter to protect other components of the access set from being damaged by the distal end of the stiffening cannula when advancing the second assembly in vivo.

33. The access set of claim 21, further comprising a third catheter for combination with the piercing device in a third assembly, the third catheter configured to accept the piercing device in a lumen of the third catheter and conceal a distal end of the piercing device with a distal-end portion of the third catheter to protect other components of the access set from being pierced by the distal end of the piercing device when advancing the third assembly in vivo.

34-66. (canceled)
